SEQUENCE LISTING (30 sequences)

SEQ ID NO: 1 - DNA, 75 bp, artificial
  Primer for bacteria
cgggaatagctcagttggtagagcacgaccttgccaaggtcggggtcgcgagttcgagtctcgtttcccgctcca

SEQ ID NO: 2 - DNA, 25 bp, artificial
  Primer for bacteria
ggagcgggaaacgagactcgaactc

SEQ ID NO: 3 - DNA, 74 bp, artificial
  Primer for bacteria
cggaagtagttcagtggtagaacaccaccttgccaaggtgggggtcgcgggttcgaatcccgtcttccgctcca

SEQ ID NO: 4 - DNA, 25 bp, artificial
  Primer for bacteria
aaggtgrtgttctaccactgaacta

SEQ ID NO: 5 - DNA, 25 bp, artificial
  Primer for bacteria
tggagcrgaagacgggattcgaacc

SEQ ID NO: 6 - DNA, 78 bp, artificial
  Primer for bacteria
tggttgcggggacaggatttgaacctgcgaccttcgggttatgagcccgacgagctaccagactgctccaccccgcga

SEQ ID NO: 7 - DNA, 16 bp, artificial
  Primer for bacteria
gcggggacaggatttg

SEQ ID NO: 8 - DNA, 25 bp, artificial
  Primer for bacteria
aaggtgrtgttctaccactgaacta

SEQ ID NO: 9 - DNA, 25 bp, artificial
  Primer for bacteria
tggagcrgaagacgggattcgaacc

SEQ ID NO: 10 - DNA, 25 bp, artificial
  Primer for bacteria
aaggtcgtgctctaccaactgagct

SEQ ID NO: 11 - DNA, 25 bp, artificial
  Primer for bacteria
ggagcgggaaacgagactcgaactc

SEQ ID NO: 12 - DNA, 25 bp, artificial
  Primer for bacteria
gtgctctaccaatctgagctaatgc

SEQ ID NO: 13 - DNA, 24 bp, artificial
  Primer for bacteria
agcataggagaatcgaactcctga

SEQ ID NO: 14 - DNA, 24 bp, artificial
  Primer for bacteria
ctgtggttttaccactaaactaca

SEQ ID NO: 15 - DNA, 25 bp, artificial
  Primer for bacteria
gacgagaatcgaactcgcgacaaca

SEQ ID NO: 16 - DNA, 25 bp, artificial
  Primer for bacteria
gctgatgttctgccattgaactaca

SEQ ID NO: 17 - DNA, 22 bp, artificial
  Primer for bacteria
agccgatgacgggaatcgaacc

SEQ ID NO: 18 - DNA, 25 bp, artificial
  Primer for bacteria
cgttggattttaccactaaaccact

SEQ ID NO: 19 - DNA, 25 bp, artificial
  Primer for bacteria
tggtgcgcaagcccggaatcgaacc

SEQ ID NO: 20 - DNA, 25 bp, artificial
  Primer for bacteria
aaacggacgctctaccactgagcta

SEQ ID NO: 21 - DNA, 25 bp, artificial
  Primer for bacteria
tggacgagtaggggatcgaacccta

SEQ ID NO: 22 - DNA, 26 bp, artificial
  Primer for bacteria
aaagccttatccgttaggccactgga

SEQ ID NO: 23 - DNA, 25 bp, artificial
  Primer for bacteria
attcgaacccgcaatctttggatta

SEQ ID NO: 24 - DNA, 25 bp, artificial
  Primer for bacteria
tggtctagtggcatgatggtaccct

SEQ ID NO: 25 - DNA, 24 bp, artificial
  Primer for bacteria
gaatcgaacccgggtcaatacctt

SEQ ID NO: 26 - DNA, 26 bp, artificial
  Primer for bacteria
aggcgagaattctaccactagaccaa

SEQ ID NO: 27 - DNA, 22 bp, artificial
  Primer for bacteria
tgcgcagtccgggaatcgaacc

SEQ ID NO: 28 - DNA, 26 bp, artificial
  Primer for bacteria
aggcgagaattctaccactagaccaa

SEQ ID NO: 29 - DNA, 22 bp, artificial
  Primer for bacteria
tgcgcagtccgggaatcgaacc

SEQ ID NO: 30 - DNA, 25 bp, artificial
  Primer for bacteria
aaggtcgtgctctaccaactgagct

(19) United States
(12) Patent Application Publication
(10) Pub. No.: US 2005/0260636 A1
(43) Pub. Date: Nov. 24, 2005

(54) Methods for the detection of biological organisms using transfer ribonucleic acids as biomarkers

(76) Inventor: X. James Li, Vienna, VA (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/088,257

(22) Filed: Mar. 24, 2005

Related U.S. Application Data
(60) Provisional application No. 60/555,683, filed on Mar. 24, 2004.

(51) Int. Cl. C12Q 1/68

(52) U.S. Cl. 435/6

(57) ABSTRACT

The present invention relates to methods and compositions for detecting nucleic acids, e.g., tRNAs in a sample. These methods can be used to detect the presence of organisms in a sample, such as gram-negative and/or gram-positive bacteria. The present invention also relates to compositions for detecting, such as a probe complex comprising: a carrier which is conjugated or bound to a at least one adaptor molecule and/or at least one polynucleotide probe molecule.

This application claims the benefit of U.S. Provisional Patent Application No. 60/555,683, filed Mar. 24, 2004, which is hereby incorporated by reference in its entirety.

### BACKGROUND

Detection of biomarkers indicative of the presence of an organism in a sample or samples is a common practice in academic research, environmental monitoring, biotechnology and medical diagnostic arenas. Commonly used biomarkers include proteins (e.g., antigens) and nucleic acids. The use of nucleic acid markers is becoming a common practice because of the sequence diversity of these markers and readily available detection methods. The types of nucleic acids that have been successfully used as biomarkers are genomic nucleic acids (deoxynucleic acids or ribonucleic acids), plasmid nucleic acids, organelle genomic nucleic acids (e.g., mitochondrial DNA), messenger RNA, and ribosomal RNA.

### DESCRIPTION OF THE DRAWING

FIG. 1 shows a tRNA-based assay format.

FIG. 2(A and B). 2A shows Probe Complex A with probe A and Poly A; 2B shows Probe Complex B with Probe B and Poly (AG).

FIG. 3 illustrates magnetic particles 13 and signal particles 15 using in another assay format.

FIG. 4 shows an alternative configuration for a probe complex.

### DESCRIPTION OF THE INVENTION

The present invention provides compositions and methods for detecting analytes. For example, the present provides methods of using transfer ribonucleic acids (tRNAs) as biological markers for rapid and sensitive diagnosis of infectious diseases. These methods are useful for detecting microorganisms in a wide variety of environments, including, e.g., bloodstream infection (BSI), catheter related bloodstream infection (CRBSI), and other types of microbial infection in subjects suspected of such infection. The methods can also be used for detecting the presence of bacteria or other microbes in clinical samples, environmental samples (such as drinking water or food), therapeutic samples, such as biologics, frozen or fresh blood transfusion units, platelet preparations for transfusion (e.g., concentrates), and blood components, and other products and materials which are to be introduced and/or placed in contact with living organisms (such as mammals and other animals).

Bloodstream bacterial infection is a serious problem that can result in high morbidity or mortality rate. A significant portion of bloodstream microbial infection is due to the infection of central venous catheter, which is commonly known as catheter related bloodstream infection or CRBSI. Diagnosis of CRBSI remains problematic and decisions to remove a catheter or treat with antibiotics are often based on clinical findings that have poor sensitivity and specificity. In fact, only a small fraction (15%) of removed catheters was proven to be the source of infection. Consequently, patients can be unnecessarily exposed to unwarranted use of antibiotics and catheter replacement, which can be associated with significant morbidity. The present invention can be used for rapid diagnosis of catheter-related bloodstream infection.

Any microorganism can be detected in accordance with the present invention, including, but not limited to bacteria, protozoa, viruses, bacteriophage, and fungi.

In an embodiment of the present invention, compositions and methods are provided for detecting transfer ribonucleic acids (tRNA). The genomes of all organisms, except for those of some viruses, encode multiple genes that code for multiple species of tRNAs, which are normally 70 to 90 nucleotide long. A bacterial species, for example, normally has 40 to 60 different tRNAs. There may be thousands of copies for each of these tRNA species in each cell, which permits more sensitive detection of the organism than genomic DNA based assay. Recent sequencing of a large number of genomes, particularly those of microorganisms, makes it possible for one to perform tRNA sequence comparison analyses and identify appropriate sequences as probes. The sequences of some tRNA species are highly conserved among even different families whereas others are distinct even within closely related species. It is therefore possible to design probes that can hybridize with a broad spectrum of bacterial species or with only one bacterial species or strain.

The present invention also provides methods of detecting a tRNA in a sample, comprising one or more of the following steps, in any effective order, e.g., contacting a sample comprising nucleic acid with a polynucleotide probe which comprises a detectable label and which is specific for a tRNA, under conditions effective for said probe to hybridize specifically with said tRNA, and detecting hybridization between said probe and said tRNA. Contacting the sample with probe can be carried out by any effective means in any effective environment. It can be accomplished in a solid, liquid, frozen, gaseous, amorphous, solidified, coagulated, colloid, etc., mixtures thereof, matrix. For instance, a probe in an aqueous medium can be contacted with a sample which is also in an aqueous medium, or which is affixed to a solid matrix, or vice-versa.

The present invention also provides methods that utilize two or more probes that hybridize to different regions of a tRNA. At least one of the probes can be used as a “capture probe” for capturing the tRNA (e.g., using a magnetic particle), and at least one probe can be marked and used as a “detector probe” which permits the detection of the captured tRNA.

Generally, as used throughout the specification, the term “effective conditions” means, e.g., the particular milieu in which the desired effect is achieved. Such a milieu, includes, e.g., appropriate buffers, oxidizing agents, reducing agents, pH, co-factors, temperature, ion concentrations, etc. When hybridization is the chosen means of achieving detection, the probe and sample can be combined such that the resulting conditions are functional for said probe to hybridize specifically to nucleic acid in said sample.

Polynucleotide probes can be designed to any desired region of a target polynucleotide. For example, one probe can be used that hybridizes with the 3′ terminal region while the other probe hybridizes with the 5′ terminal region. If more than two probes are used, then the extra probes can hybridize with any appropriate region or regions between the two terminal regions. A terminal region here means the region located at or near the terminus, e.g., within 45 nucleotides from the 3′ or 5′ terminus. It is understood that capture and detection probes may contain extensions that do not substantially hybridize with any portion of the tRNA. It is also understood that a probe may be of DNA, RNA, peptide nucleic acid, any chemical entity that permits specific base pairing, or a combination of two or more of above. It is further understood that a probe may not perfectly complement with a respective region in a tRNA. A probe can be of any length that is effective for it to hybridize with the desired target, e.g., 15 nucleotide bases or longer.

In certain embodiments, at least one capture probe can hybridize with a terminal region while at least one detection probe can hybridize with the other terminal region. This design allows at least one capture probe and at least one detection probe to be located at opposite termini, permitting more efficient hybridization.

Since a tRNA normally contains at its termini 6 to 7 base sequences that are complementary with one another thereby forming a hairpin-like secondary structure, it is desirable that one of the probes for 3′ or 5′ terminus contain little or no such sequence, which minimizes self-hybridization between the two probes.

The capture probes can be immobilized on a solid surface such as that of magnetic particles or microwell plate wells. Alternatively, the capture probes may be marked with an affinity group, i.e., a ligand, or a unique nucleic acid sequence. For example, biotin may be coupled to the terminus of a capture probe. In this case, a solid phase coated with avidin or its derivative may be used to capture the tRNA after hybridized with the capture probe.

The detection probes can be directly marked with a detectable label such as a fluorescent compound, a chemiluminescent compound, an electrochemiluminescent compound, or a quantum dot. The detection probes can also be indirectly marked with an entity, e.g., microparticles or nanoparticles that contain the labels or detection enabling molecules. Methods for preparing the label containing entities were described in U.S. Patent Application 20040018495 published Jan. 29, 2004, U.S. Provisional Application Nos. 60/532,088 and 60/532,089 filed Dec. 23, 2003, and 60/540,576, filed on Jan. 31, 2004, which are cited here for references. Moreover, the detection probes may be marked with an affinity group such as biotin or a unique nucleic acid sequence so long as it is different from that on the capture probe. The biotin on the detection probe can be subsequently detected with avidin or its derivatives conjugated with a marker such as a fluorescent compound or an enzyme, e.g., peroxidase. If the detection probe is marked with a unique nucleic acid sequence, a variety of means can be employed to detect the unique nucleic acid sequence. For example, one can design sufficiently long extension sequence so that it can be detected with polymerase chain reaction (PCR) method. The unique nucleic acid sequence extension can also serve as a linker that hybridizes with a complementary sequence conjugated to a carrier (e.g., polymers, nanoparticles or microparticles) to which a detectable marker (e.g., fluorescent compound) is attached.

Selection of probes can begin with tRNA target selection. Any tRNA with known sequence of substantial length, e.g., longer than 50 nucleotides, can be used for initial screening. The genomes for many organisms have been completely or incompletely sequenced. The sequences of these complete or incomplete genomes normally have been deposited to public databases that are readily available on the internet, e.g., the web site of the National Institutes of Health (e.g., ncbi.nlm.nih.gov). See, also, Sprinzl and Vassilenko, Nucleic Acids Res. 2005 Jan. 1; 33(Database Issue): D139-D140 (worldwide web at uni-bayreuth.de/departments/biochemie/sprinzl/trna/). For those organisms whose genomes have been completely sequenced, the sequences of tRNA or putative tRNA genes have normally been identified. For those organisms that have only been partially sequenced, tRNA genes are often not identified but can be identified through sequence similarity comparison using known tRNA sequences.

The sequence of the tRNA selected for initial screening can be compared with the entire sequence database or with a limited database, e.g., that for microorganisms. This process can be repeated for all tRNA sequences of an organism. Depending on the intended application of the tRNA-based assay, one or more tRNA targets can be selected. For example, if the assay is for detecting bacterial contamination, the assay can be designed to detect as many bacterial species as possible. Thus, the tRNAs with most homologous sequences can be selected as potential targets for detection. On the other hand, if the assay is for detecting a particular species or strain of bacteria, the tRNAs with unique sequences can be selected as potential targets. The sequence difference can be one or more nucleotides, e.g., more than one nucleotide.

Probes can be designed based on the sequences of the selected tRNAs. As mentioned above, the capture probe and detection probe can hybridize with any desired region of it, e.g., with the opposite terminal regions of a tRNA target. For the probes for detecting a particular species or strain of an organism, the unique nucleotide or nucleotides can be located in the middle region of the probe, e.g., to provide differentiation between perfectly matched and mismatched hybridization. Any probe size length can be used that is effective to specifically hybridize to the selected target, e.g., at least 15 nucleotides or more, at least 20 nucleotides or more, etc. The probes can be polynucleotides (DNA or RNA) or its variation or combination, peptide nucleic acids, or other entities that can specifically bind to desired tRNA sequences. Nucleotides comprising a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc.

The sequences of the selected probes can be further compared with all sequences in the database, and those probes that are specific or characteristic of the target tRNAs can be selected. The phrases “specific for” or “specific to” a target polynucleotide have a functional meaning that the polynucleotide can be used to identify the presence of one or more target nucleic acids in a sample and distinguish them from non-target nucleic acids. It is specific in the sense that it can be used to detect polynucleotides above background noise (“non-specific binding”). A specific sequence is a defined order of nucleotides (or amino acid sequences, if it is a polypeptide sequence) which occurs in the polynucleotide, and which is characteristic of that target sequence, and substantially no non-target sequences. A probe or mixture of probes can comprise a sequence or sequences that are specific to a plurality of target sequences, e.g., where the sequence is a consensus sequence, a functional domain, etc., e.g., capable of recognizing a family of related tRNAs. A specific polynucleotide according to the present invention can be determined routinely.

It is also possible that the probes may have significantly homologous sequences in a large genome, e.g., human genome when the probes are relatively short, e.g., less than 20 nucleotides. The presence of these sequences in the genomes of non-targeted organisms, e.g., a host organism, may not significantly interfere with a tRNA-based assay since both the capture probe and detection probe would have to bind to the genomic sequences. The selected probes are experimentally evaluated. Specific examples for probe selection are provided in the examples below.

More than one tRNA species can be targeted for certain applications. This is particularly desirable when one wishes to detect multiple organisms or strains of an organism, where even the most conserved tRNA species may not be strictly conserved among those to be detected. In this case, one may use two or more tRNA targets that represent all species to be detected. For example, multiple tRNA targets can be used, some of which are specific for Gram-negative bacteria while the others are specific for Gram-positive bacteria, in order to detect both Gram-negative and Gram-negative bacterial species in a single assay. The use of multiple tRNA targets may also improve the sensitivity of the assay even if the assay is for the detection of a single species of organism.

It is also within the scope of this invention that tRNAs are used for multiplexing assays, where multiple tRNA species are used as distinct biomarkers for different organisms, or different classes of organisms, in a single assay. For this type of applications, capture probes can be marked with the same unique extension sequence or affinity group (e.g., biotin) whereas each detection probe is marked with a distinct detectable component (e.g., fluorescent compounds with distinct emission spectra or distinct nucleic acid sequences).

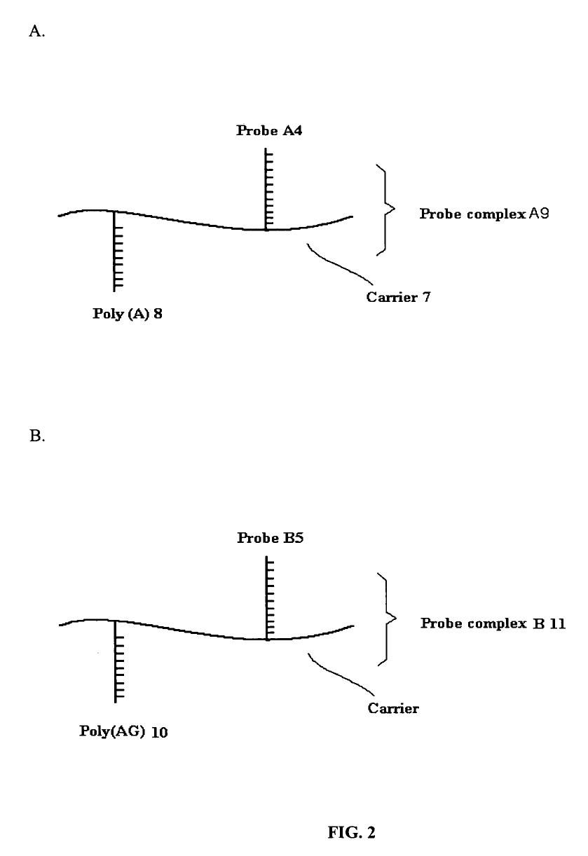

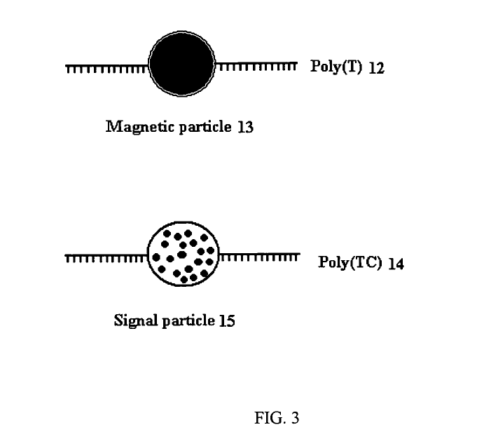

The present invention also relates to a probe complex, comprising, e.g., 1) a carrier, 2) at least one adaptor molecule, and 3) at least one polynucleotide probe molecule, wherein said adaptor comprises a specific binding region for a target substance, and said polynucleotide probe is specific for a target nucleic acid, and wherein said adaptor molecule and said polynucleotide probe are conjugated to said carrier. See, e.g., FIG. 2-4. The phrase “comprises a specific binding region for a target substance” indicates that the adaptor has a segment that is able to specifically attach to a desired substance, such as a capture moiety or a detectable label. As shown in FIG. 2, the adaptor molecule can comprise a polynucleotide (polyA or polyAG) which enables it to specifically attach to a magnetic particle to form a capture probe, or to a signal particle to form a detector probe. In this case, the adaptor is a polynucleotide that comprises a specific polynucleotide sequence which is complementary to a polynucleotide sequence attached to a magnetic particle or signal particle (as shown in FIG. 3). The sequence can be arbitrarily selected, but which typically is not present in the target nucleic acid to be detected. Specific binding regions can also comprise, e.g., binding pairs, such as biotin or streptavidin, and antibodies (including single chain).

Any suitable method for detecting tRNA can be utilized in accordance with the present invention. For example, methods of the present invention can be carried out as described in U.S. Patent Application 20040018495 published Jan. 29, 2004, which is hereby incorporated by reference in its entirety.

The topic headings set forth above are meant as guidance where certain information can be found in the application, but are not intended to be the only source in the application where information on such topic can be found.

For other aspects of the polynucleotides, reference is made to standard textbooks of molecular biology. See, e.g., Hames et al., Polynucleotide Hybridization, IL Press, 1985; Davis et al., Basic Methods in Molecular Biology, Elsevir Sciences Publishing, Inc., New York, 1986; Sambrook et al., Molecular Cloning, CSH Press, 1989; Howe, Gene Cloning and Manipulation, Cambridge University Press, 1995; Ausubel et al., Current Protocols in Molecular Biology, John Wiley & Sons, Inc., 1994-1998.

Without further elaboration, it is believed that one skilled in the art can, using the preceding description, utilize the present invention to its fullest extent. The preceding preferred specific embodiments are, therefore, to be construed as merely illustrative, and not limiting the remainder of the disclosure in any way whatsoever. The entire disclosure of all applications, patents and publications, cited above and in the figures are hereby incorporated by reference in their entirety, including U.S. Provisional Patent Application No. 60/555,683, filed Mar. 24, 2004.

### EXAMPLES

### Example 1

### Selection of Probes for the Detection of Gram-Negative or Gram-Positive Bacteria

This example describes a method for selecting appropriate polynucleotide probes for detecting a broad spectrum of bacterial species. The tRNA sequences of Escherichia coli (E. coli) strain K12 are used as the starting point. The complete genomic sequences of many organisms, including that of E. coli K12, are available from several public institutions and readily accessible through the internet. The web site of the National Center for Biotechnology Information (NCBI) lists all completed genome sequences. See, e.g., the web address at ncbi.nlm.nih.gov or ncbi.nlm.nih/genomes/MICROBES/Complete.html which lists microbes whose genomes have been completely sequenced. On this web page, it also shows the Genbank accession numbers for the sequences of these genomes. For example, the Genbank accession number for E. coli K12 is NC_{—}000913. The hyperlink of this accession number can direct one to the posted genomic sequence. Alternative, one can search the GenBank database using the accession number. The sequences of tRNAs or putative tRNAs are normally identified by the time a complete genomic sequence is posted on the web.

The genome sequence can be downloaded to a personal computer and used to identify tRNA sequences manually or by using software tools, programs or computational algorithms. The tRNA sequences can now be used to search for similar nucleotide sequences in the database using, for example, the BLAST program at ncbi.nlm.nih.gov/BLAST/. For the detection of a broad spectrum of bacterial species, tRNAs with highly homologous sequences are preferred targets for detection. Several tRNAs of E. coli K12 strain are highly homologous with the tRNAs of other Gram negative bacterial species. For example, sequence of the following tRNA-GlyGcc is highly homologous among many Gram-negative bacterial species: 5′CGGGAATAGCTCAGTTGGTAGAGCACGACCTTGCCAAGGTCGGGGTCGCGA GTTCGAGTCTCGTTTCCCGCTCCA3′ (SEQ ID NO: 1). Based on this sequence, two probes are designed as followed: Probe A-5′ AAGGTCGTGCTCTACCAACTGAGCT, and Probe B-5′ GGAGCGGGAAACGAGACTCGAACTC (SEQ ID NO: 2). Probes A and B are complementary with the 5′ and 3′ terminal regions, respectively, of tRNA-Gly_{GCC}. The sequences of both probes can be blasted again against the database to ensure that they do not share significant sequence homology with other organisms or species except for those to be detected. Probes A and B appear to be only highly homologous with certain tRNAs in Gram-negative bacteria, but not with other organisms.

If none of E. coli K12 tRNAs are significantly homologous with any tRNA in Gram-positive bacterial species, another pair of probes for the detection of Gram-positive bacterial species can be selected. The tRNA sequences of a Gram-positive bacterium with complete genomic sequence, e.g., Bacillus subtilis, can be used to blast against the sequence database as described above. A tRNA-Gly of B. subtilis has significant homology with a tRNA of many Gram-positive species. The sequence of this tRNA-Gly is as follows: 5′CGGAAGTAGTTCAGTGGTAGAACACCACCTTGCCAAGGTGGGGGTCGCGG GTTCGAATCCCGTCTTCCGCTCCA3′(SEQ ID NO: 3). Two probes are designed based on the sequence of this tRNA:

Probe G1: 5′AAGGTGRTGTTCTACCACTGAACTA (SEQ ID NO: 4), and Probe G2: 5′TGGAGCRGAAGACGGGATTCGAACC (SEQ ID NO: 5), where “R” stands for a degenerative position with nucleotide bases A and G in order to cover more Gram positive bacterial species. Again, Probe G1 and G2 hybridize with the 5′ and 3′ terminal regions of the tRNA, respectively.

Probes A and B can be used to detect a substantial number of Gram-negative bacterial species whereas Probes G1 and G2 can be used to detect a substantial number of Gram-positive bacterial species. Moreover, a combination of Probes A, B, G1 and G2 should permit the detection of a substantial number of Gram-negative and -positive bacterial species.

### Example 2

### Selection of Probes for the Detection of a Particular Bacterial Species

This example describes a method for selecting probes for the detection of a particular bacterial species. Point mutation, deletion mutation or insertion mutation at an appropriate location of a tRNA target of a particular species or strain can be used for differentiating the species to be detected from others. Generally, deletion and insertion mutations are preferred, but they are less frequent.

This example discloses a method for selecting probes for the detection of Bacillus anthracis, which causes Anthrax disease. Sequence comparison using methods described above identified at least two potential tRNA targets, which has at least one point mutation at favorable position, e.g., in the middle of a potential probe, when compared to B. anthracis's closest relatives: B. cereus and B. thuringiensis. One example is one of the tRNAs for the amino acid methionine, which is designated as tRNA-Met-6 here, whose sequence is as follows: 5′TGGTTGCGGGGACAGGATTTGAACCTGCGACCTTCGGGTTATGAGCCCGAC GAGCTACCAGACTGCTCCACCCCGCGA (SEQ ID NO: 6). The underlined nucleotide is the point mutation, which can be used to differentiate B. anthracis from its closely related species. A probe complementary with 16 nucleotides of the 5′ terminal tRNA sequence (GCGGGGACAGGATTTG) (SEQ ID NO: 7) with the point mutation in the middle region can be used as the detection probe or, preferably, as the capture probe. In order to minimize non-specific hybridization, the point mutation is preferably located in the central region or its vicinity and the probe is relatively short, e.g., fewer than 20 nucleotides. Preferably, the probe is a peptide nucleic acids (PNA), which may provide stronger binding between the probe and target tRNA. Alternatively, a locked nucleic acid (LNA) may be used in the position complementary with the point mutation. LNA contains a 2′-O, 4′-C methylene bridge, which restricts the flexibility of the ribofuranose ring and therefore creates highly stable complementary duplex. An increase in Tₘ (melting point) of upto 8° C. for each LNA monomer in a probe had been observed.

The other probe can be based on the 3′ terminal sequence of the tRNA. Although the 3′ sequence of this tRNA is identical among B. anthracis, B. cereus and B. thuringiensis, the difference in 5′ probe is sufficient for specific detection of B. anthracis under appropriate conditions. If the 5′ probe described above is used for capturing, only the B. anthracis tRNA can be captured, which will result in specific detection even though the 3′ probe is nonspecific. If the 5′ probe is used for detection and the 3′ probe for capturing, only the B. anthracis tRNA can be detected even though tRNAs from all three species are captured.

### Example 3

### A Format of a tRNA-Based Assay

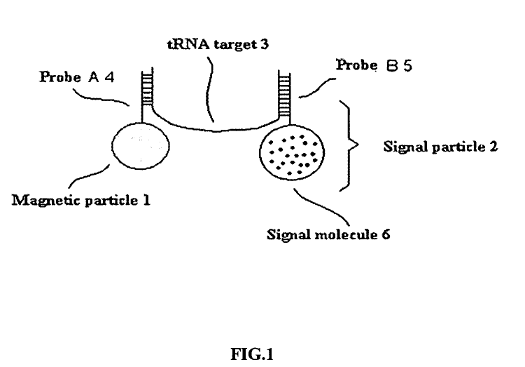

As shown in FIG. 1, this assay can use two key components, the magnetic particles 1 and signal particles 2. The magnetic particles 1 are coated with a probe specific for one region of the tRNA target 3, and the signal particles 2 are coated with another probe specific for another region of the tRNA target 3. The magnetic particle 1 can be referred to also as a capture probe, and the signal particle 2 can be referred to as a detector probe.

The magnetic particles 1 can be of any suitable size that is effective for it to be captured, e.g., at least about 0.1 micrometer or more in diameter, e.g., at least about 1 micrometer or more in diameter, or at least about 3 micrometer or more in diameter, etc.

The signal particles can be associated with a signaling molecule 6. The signaling molecule can also be referred to as a detectable label. Any signaling molecule that is capable of detection can be used, including those that generate, or enable the generation of, chemiluminescence, electrochemiluminescence, fluorescence, or color. One specific example for a chemiluminescent signaling molecule is acridinium. The signal particles 2 can be of any suitable size or composition that enable it to be detected, including, e.g., less than about 10 micrometers in diameter, less than about 5 micrometers in diameter, or less than about 2.5 micrometers in diameter, etc. The signal particles can be associated with signal molecules in any effective way, e.g., encapsulated with signal molecules 6, covalently attached thereto, coordinately attached thereto, etc. The more signal molecules a signal particle has, the better sensitivity the assay may have.

The probes for the tRNA targets may be selected as described in Example 1. Probes A and B, which are selected according to Example 1, are used in this example simply for the purpose of illustrating the assay. While probe A 4 and probe B 5 preferably hybridize with a tRNA target of Gram negative bacteria. As described in Example 1, probes A 4 hybridizes with the 5′ terminal region of the tRNA target 3 whereas probes B 5 hybridize with the 3′ region of the tRNA target 3.

The magnetic particles are coated with probes A 4 whereas the signal particles are coated with the probes B 5. A magnetic particle or signal particle may be coated with one probe, e.g., probe A or probe B, or with a mixture of more than one probe, e.g., probes A and G1 for magnetic particles or probes B and G2 for signal particles. In the case where a particle is coated with only one type of probe, a mixture of particles coated with different probes can be used together for detecting more than one tRNA targets.

As illustrated in FIG. 1, target tRNAs are captured onto the magnetic particles through specific hybridization between the Probe A 4 on the magnetic particles and tRNA target 3 under appropriate conditions. Hybridization conditions are well known in the art. See, e.g., Sambrook et al., Molecular Cloning, CSH Press, 1989; Ausubel et al., Current Protocols in Molecular Biology, John Wiley & Sons, Inc., 2004. After washing with appropriate solution to remove non-target components in the sample, the magnetic particles 1, which are now labeled with the target tRNA 3, are incubated with the signal particles 2, which results in the formation of a magnetic particle-tRNA-signal particle sandwich complex. After washing to remove unbound signal particles 2 using a magnet, the bound signal particles 2 are measured through the associated signal molecules 6 using appropriate instrument, e.g., a luminometer for the measurement of chemiluminescent compounds.

### Example 4

### Another Format for a tRNA-Based Assay

This example describes another assay format, which is a modified version of the assay format described in Example 3. The basic feature is still the magnetic particle-tRNA-signal particle complex sandwich structure. However, the tRNA-hybridizing probes are not directly conjugated onto the magnetic particle or signal particles. Rather, the probes are conjugated to a carrier, resulting in an intermediate complex, as shown in FIGS. 2A and 2B. Any material can be used as a carrier. Examples for the carriers include, but are not limited to, a linear polymer (e.g., polylysine), branched polymer (e.g., dendrimer), nanoparticles (e.g., gold particles, polystyrene particles) or microparticles and other macromolecules that permit the coupling of multiple oligonucleotides. Poly-D-lysine is used here as a specific example. Nanoparticles with functional groups on their surface can also be used as the carrier. A 50 to 100 nanometer particle can have thousands of functional groups (e.g., carboxyl group, primary amine) on its surface, thereby permitting the labeling of multiple numbers of affinity groups and probes.

The probe conjugates are also referred to as probe complex in this application. In general, a probe complex can comprise two types of affinity groups, both of which are coupled to the carrier: one or more probes, e.g., 1) Probe A described in Example 3, that specifically hybridizes with one region of the tRNA targets and 2) one or more second affinity groups, e.g., biotin or an oligonucleotide, that serve as adaptors, which can specifically bind to either the magnetic particles or signal particles but preferably not both.

The second affinity groups are referred to as adaptor in this application. An adaptor is utilized to attach a detector probe to the carrier. In this example, probe A 4 is conjugated to the carrier 7 along with an adaptor, which is a poly (A)ₙ polynucleotide 8 (FIG. 2A). The poly A can be of any effective size, e.g., at least 20 nucleotides long, where the string of As serve as a binding partner for a polyT that contains the detector moiety. Similarly, probe B 5 (FIG. 2B) is conjugated to the carrier along with another adaptor, which is another polynucleotide, poly (AG)ₙ 10. Poly (AG) 10 can be of any effective size, e.g., at least 20 nucleotides long. Each probe complex may comprise one or more tRNA target specific probes. While there may be large number of probes for large number of tRNA targets, there are only two adaptors: one for magnetic particles and one for signal particles.

As illustrated in FIG. 3, this assay uses the magnetic particles and signal particles as well. The characteristics of the magnetic particles are described in Example 3, but are coated with an affinity group that can specifically bind to one of the adaptors, e.g., polynucleotide (T), which is complementary with poly (A) in probe complex A 9 (FIG. 2A). The characteristics of signal particles are also described in Example 3, but are coated with another affinity group, e.g., poly (TC), which is complementary with poly (AG) in probe complex B 11 (FIG. 2B).

The use of probe complexes in this assay has several advantages. First, more than one probe molecules and adaptor molecules can be conjugated to a carrier molecule or entity, which can increase the binding efficiency between a probe and its target tRNA and between an adaptor and magnetic particles or signal particles. Second, the probe complexes can be added to the sample lysis solution, which allows lysis and target tRNA hybridization simultaneously or sequentially. Third, hybridization between target tRNA and the probe may be more efficient since probe complexes are present in solution, i.e., not on a solid phase. It also permits the use of adaptors that in turn allows the use of magnetic particles and signal particles that are not specific for assay or analyte targets, e.g., tRNAs.

To perform detection, the sample is first lysed in an appropriate buffer such as 5 Molar guanidine isothiocyanate in 0.25 Molar sodium phosphate buffer (pH 7.4), which contains appropriate amounts of probe complexes. For example, probe complex A 9 and probe complex B 11 are included for the detection of Gram-negative bacteria. Further inclusion of probe complex G1 and G2 would enable the detection of Gram-positive bacteria as well.

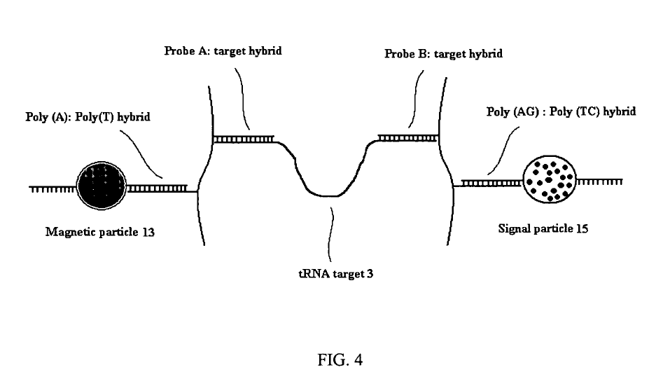

After incubation under appropriate conditions, e.g., 80° C. for 10 minutes to denature tRNA targets and 50° C. for probe hybridization, a poly A-tRNA-poly AG complex is formed if and only if the target tRNA is present, as shown in FIG. 4. Sufficient amounts of magnetic particles, which are coated with poly T, are then added to the lysed sample followed by incubation at appropriate temperature for an appropriate period of time. All poly A containing probe complexes, including those that are hybridized with tRNA targets, are captured onto the magnetic particles. If there is target tRNA in the sample, some of the magnetic particles will be labeled with poly AG adaptor. After washing to remove the unbound probe complex, which contains the poly AG adaptor, the magnetic particles are then incubated with the signal particles, resulting in the formation of magnetic particle-tRNA-signal particle complex as shown in FIG. 4. After washing to remove unbound signal particles, the sample is measured for bound signal molecules using appropriate instrument, e.g., luminometer for chemiluminescent compounds. The presence of bound signal molecules is indicative of the presence of target tRNA and hence the organism.

It is within the scope of this invention that the assay formats are combinations of those described in Examples 3 and 4. For example, the magnetic particles may be directly coated with target tRNA binding probes while a probe complex is used to bridge the target tRNA and signal particles. Alternatively, the signal particles are coated with tRNA binding probes whereas a probe complex is used to bridge the target tRNA and signal particles. It is also within the scope of this invention that no signal particles are used in the assay. The signal molecules, e.g., acridinium, may be directly conjugated to the probes that hybridize with one region of the tRNA targets. Alternatively, a component that enables signal generation, e.g., peroxidase, can be directly conjugated to one of the probes. Signal molecules or the component that can generate signal can be indirectly conjugated to the probe through a carrier to form a probe complex with detectable markers.

### Example 5

The following are specific examples for making the probe complexes, magnetic particles and signal particles.

### Preparation of Probe Complexes

Refer to FIGS. 2A and 2B, which illustrates the schematic structure of a probe complex. In this example, the probe complex is a poly-D-lysine molecule coupled with two distinct oligonucleotides, one for particle binding (the adaptor) and another for tRNA binding (probe). Here, oligonucleotides are referred to as adaptor or probes since both are oligonucleotides. The molecular weight of poly-D-lysine may need to be determined experimentally. A preferred starting size is between 100 to 150 lysine residues per poly-D-lysine molecule, however, any effective size can be used.

The oligonucleotides are first activated with DSS [suberic acid bis (N-hydroxy succinimide ester)] and then coupled to poly-D-lysine with the following molar ratio: adaptor:probe:polylysine=10:10:1. If the ratio of adaptor:probe:polylysine is 10:10:1 and the coupling efficiency is 50%, each polylysine molecule should have, on average, 5 adaptors and 5 probe molecules. This ratio, however, can be adjusted, preferably according to the results of optimization experiments, coupling efficiency, tRNA targets and assay requirements.

The oligonucleotides are synthesized with an amine derivative at its 5′ or 3′ terminus. Many vendors such as Integrated DNA Technologies (Coralville, Iowa) can synthesize these oligonucleotides, which are dissolved in 10 mM HEPES buffer, pH 7.5 at a concentration of 1 nanomole per microliter.

Adaptor and probe are mixed, activated and then simultaneously conjugated to polylysine. The molar ratio of adaptor to probe can be adjusted. To perform oligonucleotide activation reaction, 10 mmoles each of adaptor and probe at 1:1 ratio are mixed with 4 microliters of 1 M HEPES, pH 7.5, 2 microliters of 0.5 M EDTA, 2 microliters of 0.5 N NaCl and 66 microliters of DSS [suberic acid bis (N-hydroxy succinimide ester)]; DSS should be prepared in DMSO (10 mg/mL) immediately before use. The reaction mix is incubated at room temperature for 25 minutes and then extracted three times with n-butanol to remove free DSS. The oligonucleotide pellet is dried under a vacuum for 15 minutes.

The activated oligonucleotides are then suspended with 100 microliters of 100 mM HEPES buffer containing 2 nanomoles of poly-D-lysine (average molecular weight=22 kDa). After incubation at room temperature for 2 to 3 hours, the free primary amines on polylysine are preferably inactivated, e.g., acetylated. Acetylation can be performed as follows: to the conjugation reaction, add 50 microliters of 1 mole/Liter acetic acid in 100 mM HEPES, 200 microliters of 1 mole/Liter carbodiimide [1-Ethyl-3-(3-dimethyl-aminopropyl)-carbodiimide hydrochloride, EDC] freshly prepared in 100 mM HEPES buffer, and 100 microliters of 2 moles/Liter NHS(N-hydryl succimide) in DMSO. Mix and incubate for 2 hours at room temperature. The probe complex can be purified using one of several well-established means, e.g., ion exchange chromatography or size exclusion chromatography or centrifugation through membrane with certain size pores. The amounts of conjugated oligonucleotides can be estimated using spectrophotmetry.

### Preparation of Magnetic Particles

As diagramed in FIG. 2, capture particles are magnetic particles covalently coated with polydeoxynucleotide T (poly T) via BSA (bovine serum albumin); the lysine residues on BSA are used for poly T conjugation. Preparation of this component was accomplished in three steps: coating of magnetic particles with BSA, activation of poly T with a homobifuntional NHS ester, and coupling of activated poly T to BSA on magnetic particles. These steps are briefly described below:

Step 1—Coating of magnetic particles with BSA: 2×10⁹ carboxylated Magnetic particles (approximately 3 micrometer in diameter) are washed three times with 0.1 M MES, pH 5.0 and then leave the particles as a wet cake. 40-60 mg of carbodiimide [1-Ethyl-3-(3-dimethyl-aminopropyl)-carbodiimide hydrochloride, EDC] is dissolved in 1.0 mL of cold 0.1 M MES buffer and immediately added to the magnetic particles. The particles are then suspended. After an addition of 20 mg of NHS in 0.1 mL of DMSO, the particles are rotated at room temperature for 30 minutes, washed three times with cold 0.1 M MES buffer, and suspended again in 0.775 mL of cold MES buffer. 0.225 mL of BSA (50 mg/mL in 25 mM MES buffer) is added to the particles. The reaction mix is incubated at room temperature with rotation for 2 to 4 hours, or overnight at 2-8° C. The particles were washed 5 times with 10 mM HEPES buffer, pH 7.5, and then suspended in 1.0 mL of 100 mM HEPES buffer with the same pH. The buffer solution was removed immediately before poly T conjugation (see Step 3).

Step 2—Activation of poly T: The oligonucleotide was synthesized with an amine derivative at its 5′ end and activated with DSS in the same way as for adaptor and probe for conjugation to poly-D-lysine for preparing probe complex. Normally at least 10 nanomoles of poly T is needed for 109 particles. Therefore, approximately 20 nanomoles of poly T is needed for 2×10⁹ particles.

Step 3—Coupling of activatedpoly T to BSA coated magnetic particles: The activated poly T pellet is dissolved in 200 microliters of cold 100 mM HEPES buffer and immediately added to the BSA coated magnetic particles prepared as described in step 1. The particles are then rotated at room temperature for about 2 hours, washed five times with TBST (50 mM Tris-HCl, pH 7.4, 150 mM NaCl, 0.5% Tween 20, and 0.1% Proclin 300), and finally stored in 1.0 mL TBST. Coupling efficiency can be determined by measuring the relative amounts of poly T in the solution at the beginning and end of the conjugation process.

### Preparation of Signal Particles

Refer to FIG. 3, where a schematic illustration of a signal particle is presented. Signal particles are coated or, preferably, encapsulated with signal molecules, e.g., acridinium or europium. In addition, the signal particles are also coated with an adaptor binding oligonucleotide poly (TC).

A number of methods for coating or encapsulating signal molecules are known to the skilled people in the art. For example, europium chelate or oxide can be encapsulated in microparticles or coated with a polymer layer with functional groups, e.g., carboxyl group. The functional group can be used for conjugating poly (TC) on to the particles. The encapsulated or embedded europium chelate or oxide can be detected using a fluorometer or an electrochemiluminescence detector.

### Example 6

### Use of Microbial tRNA Markers for Rapid Diagnosis of Catheter Related Bloodstream Infection (CRBSI)

Selection of Target Organisms and Detection Probes

A diagnostic test for CRBSI can be designed that detects a broad spectrum of microbial species that are commonly associated with CRBSI. According to a publication by Raad (Lancet 1998; 351: 893-98) cited here solely as a reference, microbes commonly associated with CRBSI can be divided into three categories: A—organisms that dwell on human skin, B—organisms that contaminate the hands of medical personnel, and C—organisms that are emerging as pathogens for CRBSI. Table 1 lists examples for each category. These organisms are listed here solely as examples.

TABLE 1
CRBSI associated microbes and their genomic sequence status

| CATEGORY | # | Microbe | Probe Pairs |
|---|---|---|---|
| A | 1 | Staphylococcus aureus | G1/G2 |
| | 2 | Staphylococcus epidermidis | G1/G2 |
| | 3 | Bacillus species | G1/G2 |
| | 4 | Corynebacterium species | Cor1/Cor2 |
| B | 5 | Pseudomonas aeruginosa | A/B |
| | 6 | Enterococcus faecalis | Ent1/Ent2 |
| | 7 | Klebsiella pneumoniae | A/B |
| | 8 | Serratia marcescens | A/B |
| | 9 | Stenotrophomonas maltophilia | A/B |
| | 10 | Candida species | Y1/Y2 |
| | 11 | Acinetobacter species | |
| C | 12 | Mycobacterium species | Myco1/Myco2 |
| | 13 | Achromobacter species | |
| | 14 | Micrococcus species | |
| | 15 | Malassezia furfur | |
| | 16 | Rhodotorula species | |
| | 17 | Fusarium species | |
| | 18 | Trichosporon species | |
| | 19 | Hansenula anomala | |

Selection of probes for those organisms begins with the selection of appropriate tRNA targets. Several potential probes are listed in Table 2. It is understood that the probes listed in Table 2 serve only as examples and that probes for different tRNA targets can be designed and used for detecting CRBSI.

Probe pairs A/B and G1/G2 are highly conserved among G− and G+bacteria, respectively. However, Corynebacterium species, Enterococci and Mycobacteria are fairly unique compared to other bacterial species in categories A and B. Therefore, additional probe pairs, Cor1/Cor2, Ent1/Ent2 and Myco1/Myco2, are designed for Corynebacterium species, Enterococci and Mycobacteria, respectively. Since a significant number of CRBSI cases are resulted from the infection of Candida species, a pair of probes (Y1/Y2) based on a S. cerevisiae tRNA-Gly sequence are also designed. Blasting the incomplete genome sequence of Candida albicans revealed that tRNA species with sequence domains highly homologous to Y1 and Y2 exist in C. albicans as well. It is likely that probe pair Y1/Y2 can substantially hybridize with one or more tRNA targets of C. albicans and its closely related Candida species. Preferably, probes specific for Candida albicans and related fungi species are designed using sequence database of C. albicans or related fungi species. The probes can be further analyzed to ensure that there are no human genomic sequences, or preferably ribonucleic acid sequences, that are substantially complementary with the probes.

TABLE 2
Probe sequences and targeted organisms

| | | | |
|---|---|---|---|
| Pair | Probe | Sequence | Targeted Organisms |

| | | | | |
|---|---|---|---|---|
| 1 | G1 | AAGGTGRTGTTCTACCACTGAACTA | (SEQ ID NO: 8) | Staphylococcus species, |
| | | (R = A or G) | | Bacillus species, |
| | G2 | TGGAGCRGAAGACGGGATTCGAACC | (SEQ ID NO: 9) | |
| | | (R = A or G) | | |
| 2 | A | AAGGTCGTGCTCTACCAACTGAGCT | (SEQ ID NO: 10) | P. aeruginosa, E. faecalis, |
| | | | | K. pneumoniae, S. marcescens, |
| | B | GGAGCGGGAAACGAGACTCGAACTC | (SEQ ID NO: 11) | S. maltophilia |
| 3 | Cor1 | GTGCTCTACCAA(T/C)TGAGCTAATGC | (SEQ ID NO: 12) | Corynebacterium species |
| | Cor2 | AGCATAGGAGAATCGAACTCCTGA | (SEQ ID NO: 13) | |
| 4 | Ent1 | CTGTGGTTTTACCACTAAACTACA | (SEQ ID NO: 14) | Enterococci |
| | Ent2 | GACGAGAATCGAACTCGCGACAACA | (SEQ ID NO: 15) | |
| 5 | Myco1 | GCTGATGTTCTGCCATTGAACTACA | (SEQ ID NO: 16) | Mycobacteria |
| | Myco2 | AGCCGATGACGGGAATCGAACC | (SEQ ID NO: 17) | |
| 6 | Y1 | CGTTGGATTTTACCACTAAACCACT | (SEQ ID NO: 18) | Sacharomyces species, Candida |
| | | | | species, Streptomyces species |
| | Y2 | TGGTGCGCAAGCCCGGAATCGAACC | (SEQ ID NO: 19) | |

Performance of the assay can be evaluated using some or all of the microbial species listed in Table 1 as described, e.g., in U.S. Patent Application 2004/0018495 or in other available assay formats. Clinical performance of the assay can be evaluated using clinical samples in clinical settings as prescribed in NCCLS guidance or other protocols commonly practiced in the field of art.

### Example 7

### Use of Microbial tRNA Markers for Rapid Diagnosis of Microbial Contaminated Platelet Preparations for Transfusion

Microbial, particularly bacterial, contamination in platelets and, to a less extent, red cells, is one of the most hazardous risks associated with transfusion of blood components. Such a hazard can be prevented or minimized with an effective bacterial detection test. The microbial test used for detecting contamination in platelets is substantially similar to that used for detecting CRBSI described above. Same probes or substantially similar probes can be used for the detection of microbial contamination in platelets. Detection technology that is the same or different from that used in CRBSI test may be used for the detection of microbial contamination in platelets.

### Example 8

### Use of tRNA Markers for Rapid Diagnosis of Protozoa Infection in Human

Infection of protozoa in human is common in tropical and subtropical regions. Diseases caused by protozoa include Malaria, Leishmaniasis, African sleeping sickness and Chagas' disease. These disease-causing protozoa are genetically distinct from human, bacteria or fungi. Protozoa tRNAs are therefore appropriate targets for the detection of protozoa infection in human.

This example is for the detection of Leshmaniasis, i.e., infection of Leishmania. Selection of the probe sequences are based on the genomic sequence of Leishmania major. tRNA from other Leishmania species and for other protozoa can also be utilized so that probes specific for multiple Leishmania species can be selected. One or more pairs of the probes can be used in an assay.

The ability of the probes to specifically detect Leishmania can be evaluated using cultured protozoa and/or clinical samples in clinical setting. Again, detection technology that is the same or different from that used in CRBSI test may be used for the detection of Protozoa.

TABLE 3
Nucleic Acid Probes for the Detection of Leishmaniasis

| | | | |
|---|---|---|---|
| Pair | Probe | Sequence | Targeted tRNA |

| | | | | |
|---|---|---|---|---|
| 1 | Ala-1 | AAACGGACGCTCTACCACTGAGCTA | (SEQ ID NO: 20) | One of the alanine tRNA |
| | Ala-2 | TGGACGAGTAGGGGATCGAACCCTA | (SEQ ID NO: 21) | |
| 2 | Arg-1 | AAAGCCTTATCCGTTAGGCCACTGGA | (SEQ ID NO: 22) | One of the arginine tRNA |
| | Arg-2 | ATTCGAACCCGCAATCTTTGGATTA | (SEQ ID NO: 23 | |
| 3 | Gly-1 | TGGTCTAGTGGCATGATGGTACCCT | (SEQ ID NO: 24) | One of the glycine tRNA |
| | Gly-2 | GAATCGAACCCGGGTCAATACCTT | (SEQ ID NO: 25) | |
| 4 | Gly-3 | AGGCGAGAATTCTACCACTAGACCAA | (SEQ ID NO: 26) | One of the glycine tRNA |
| | Gly-4 | TGCGCAGTCCGGGAATCGAACC | (SEQ ID NO: 27) | |
| 5 | His-1 | AGGCGAGAATTCTACCACTAGACCAA | (SEQ ID NO: 28) | One of the histidine tRNA |
| | His-2 | TGCGCAGTCCGGGAATCGAACC | (SEQ ID NO: 29) | |

1. A method of detecting a tRNA in a sample, comprising:
contacting a sample comprising nucleic acid with a plurality of polynucleotide probes specific for a tRNA,
under conditions effective for said probes to hybridize specifically with said tRNA,
wherein each probe is specific for a different region of the same tRNA, and
detecting hybridization between said probe and said tRNA.

2. A method of claim 1, wherein at least one probe is attached to a magnetic particle and at least one probe is attached to a detectable label.

3. A method of claim 1, wherein said at least one probe is attached to microparticles which comprise a detectable label.

4. A method of claim 1, where said polynucleotide probe is a probe complex, comprising:
a carrier, at least one adaptor molecule, and at least one polynucleotide probe molecule,
wherein said adaptor comprises a specific binding region for a target molecule, and said polynucleotide probe is specific for a target nucleic acid, and
wherein said adaptor molecule and said polynucleotide probe are conjugated to said carrier.

5. A method of claim 1, wherein probes specifically detect gram-negative, but not gram-positive bacteria.

6. A method of claim 1, wherein said tRNA is a bacterial tRNA.

7. A method of claim 4, wherein said specific binding region for a target molecule comprises a polynucleotide comprising a polynucleotide sequence.

8. A method of claim 4, wherein a capture probe complex and detector probe complex are utilized.

9. A method of claim 8, wherein said capture probe complex comprises a specific binding region comprising a polynucleotide sequence which is complementary to a polynucleotide sequence attached to a magnetic particle.

10. A method of claim 8, wherein said detector probe complex comprises a specific binding region comprising a polynucleotide sequence which is complementary to a polynucleotide sequence attached to a signaling particle.

11. A method of claim 9, further comprising contacting said capture probe complex with said polynucleotide attached to said magnetic particle.

12. A method of claim 15, further comprising contacting said detector probe complex with said polynucleotide attached to said signaling particle.

13. A method of claim 1, wherein said sample comprises blood to test for a catheter-related bloodstream infection.

14. A method of claim 1, wherein said sample comprises a platelet preparation for transfusion to test for microbial contamination.

15. A method of claim 1, wherein said sample is a human tissue to test for protozoa infection.

16. A probe complex, comprising:
a carrier, at least one adaptor molecule, and at least one polynucleotide probe molecule,
wherein said adaptor comprises a specific binding region for a target molecule, and said polynucleotide probe is specific for a target nucleic acid, and
wherein said adaptor molecule and said polynucleotide probe are conjugated to said carrier.

17. A probe complex of claim 16, wherein said wherein said specific binding region for a target molecule comprises a polynucleotide comprising a polynucleotide sequence.
